(19) United States
(12) Plant Patent Application Publication
(10) Pub. No.: US 2013/0019355 P1
(43) Pub. Date: Jan. 17, 2013

(54) Hop plant named 'HORT7709'

(50) ;

(71) Applicant: Ron Beatson, Motueka (NZ)

(72) Inventor: Ron Beatson, Motueka (NZ)

(73) Assignee: The New Zealand Institute for Plant and Food Research Limited, Auckland (NZ)

(21) Appl. No.: 13/135,815

(22) Filed: Jul. 15, 2011

(51) Int. Cl. A01H 5/00

(52) U.S. Cl. PLT/236

(57) ABSTRACT

A new and distinct hops plant is described. The cultivar results out of a selection from a population of seedlings derived from the open pollination of a tetraploid seedling selection 95-28-24 (not patented). The cones are of a small size, oval shape, ripen mid-late season, and have slightly open bracts. The cultivar has been shown to have a unique chemistry profile; of particular interest is the high yield of oil per gram of alpha acid.

### GENUS AND SPECIES OF PLANT CLAIMED

Humulus lupulus L.

### VARIETY DENOMINATION

‘Hort7709’

### BACKGROUND TO THE INVENTION

Seed was obtained from open pollination of the unreleased tetraploid seedling selection 95-28-24 (not patented) in the 1996/97 season. 95-28-24 was identified as a tetraploid seedling from the open pollination of ‘Liberty’ (not patented). Triploid seedlings obtained from this open pollination were grown in a nursery at the Motueka, New Zealand in the 1997/98 season; ploidy level was determined by flow cytometry. The resulting seedlings were then planted out in the field and grown in the 1998/99 season. The seedling, originally designated the breeder code 97-77-09 and later named ‘Hort7709’, was selected during the 1998/99 season on the basis of its agronomic performance and chemistry profile.

### SUMMARY OF THE INVENTION

A new and distinct hops plant is described. The cones of ‘Hort7709’ are of a small size, oval shape, ripen mid-late season, and have slightly open bracts. The cultivar has been shown to have a unique chemistry profile; of particular interest is the high yield of oil per gram of alpha acid.

‘Hort7709’ was relocated to the seedling selections repository in winter 1999 where it underwent performance monitoring in the 1999/2000 season. During this time it was clonally propagated. ‘Hort7709’ underwent evaluation in a small plot replicated trial with nine other promising aroma selections for 3 seasons, from 2000/01 to 2002/03.

On the basis of its performance for agronomic, chemistry and brewing characteristics, it was decided to advance ‘Hort7709’ to large-scale testing. In late winter 2002 a 150-plant plot was established at Motueka, New Zealand and over the next eight seasons the cultivar underwent extensive brewing, agronomic, and chemistry evaluations. In addition to this testing a 500-plant grower trial was established in the Nelson Province in 2005. ‘Hort7709’ is maintained at Motueka as a 150-plant plot where it has undergone observations for uniformity. All plants have been found to be true to type, that is, no off types have been observed. Under New Zealand growing conditions ‘Hort7709’ is distinguished from the known parental cultivar 95-28-24 and other cultivars of common knowledge by the following characteristics along with its unique chemistry profile and brewing characteristics.

95-28-24: the maternal parent 95-28-24 is tetraploid whereas ‘Hort7709’ is triploid, the ploidy level has been confirmed by flow cytometry.

‘NZ Hallertauer’ (not patented): when mature ‘Hort7709’ vines have an overall club-shape with a medium head volume and a late harvest season, while ‘NZ Hallertauer’ vines are cylindrical in shape with a low head volume and an early harvest season.

‘Pacifica’ (not patented): when mature ‘Hort7709’ vines have an overall club-shape, a medium number of thorns, and a reddish-purple stripe, while ‘Pacifica’ vines are cylindrical in shape, have very few thorns and a green stripe.

### BRIEF DESCRIPTION OF THE DRAWINGS

The accompanying photographs show typical specimens of the plant habit, flowers, and leaves of the new cultivar as depicted in colours as nearly true as is reasonably possible to make the same in colour illustration of this character.

FIG. 1: Five year mean of at harvest chemistry profiles for ‘Hort7709’ and two comparator cultivars ‘NZ Hallertauer’ (not patented) and ‘Pacifica’ (not patented). Trials and testing was conducted in Motueka, New Zealand.

FIG. 2: Mature fully expanded leaves of ‘Hort7709’ (A) and ‘NZ Hallertauer’ (B).

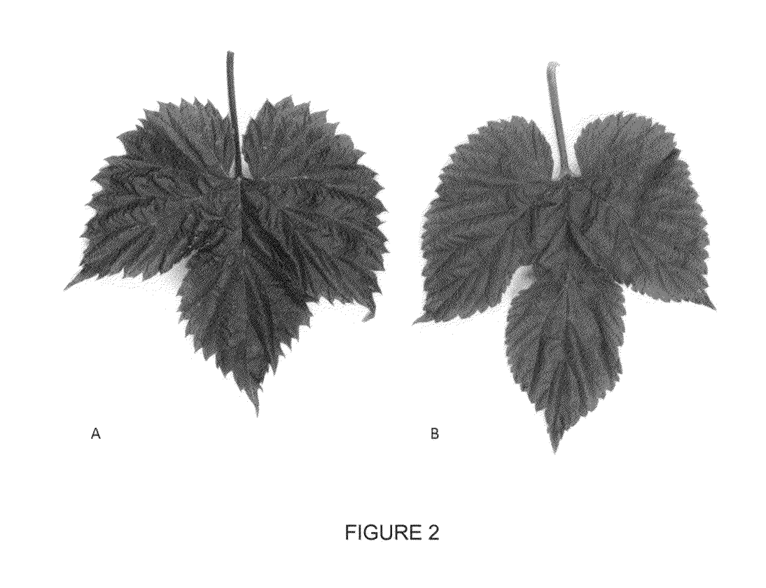

FIG. 3: Cones of ‘Hort7709’ (A) and ‘NZ Hallertauer’ (B).

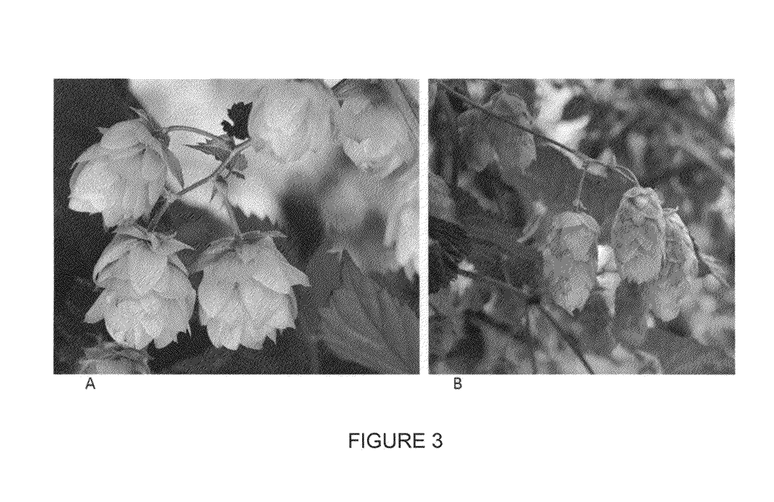

FIG. 4: Close up of mature vines ‘Hort7709’ (A) and ‘NZ Hallertauer’ (B).

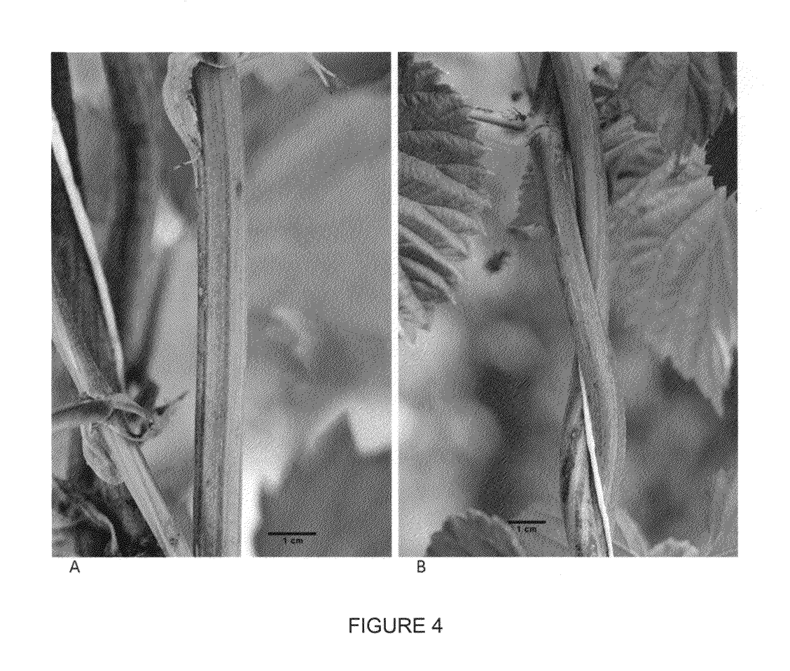

### DETAILED DESCRIPTION

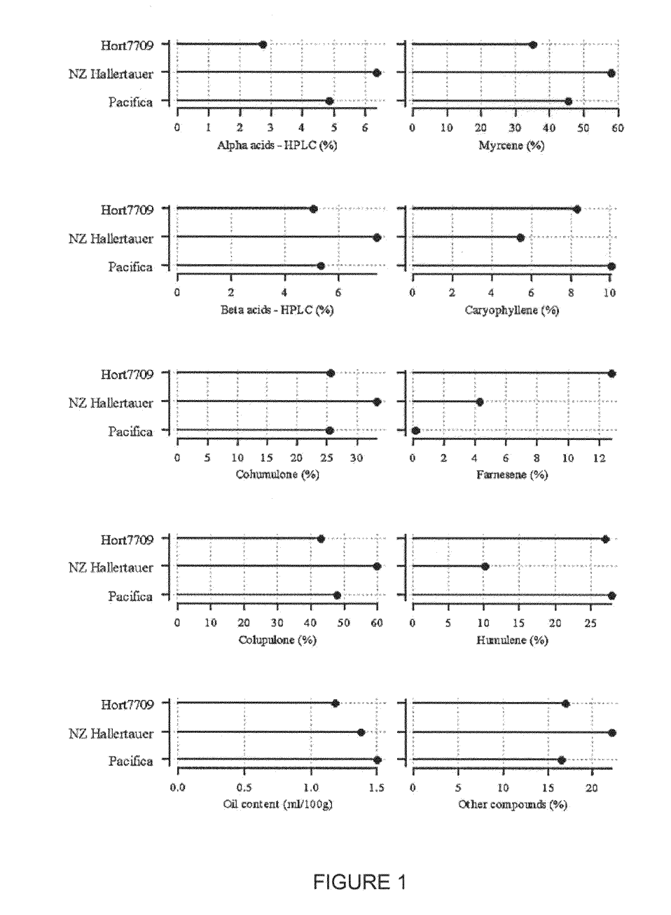

The following is a description of the new cultivar with colour terminology in accordance with The Royal Horticultural Society Colour Charts (R.H.S.C.C.) 2001 edition. The specimens described were grown at Motueka, New Zealand. The observations were made over the 2009-2011 seasons.
Plant form and vigour: Plants are of a normal growth type, are club-shaped when mature and produce a moderate yield (average of approximately 1700 kg/ha). The vine is thick and very vigorous but is relatively slow to establish, training is delayed until later in the season. Hop plants are usually trained in late October-early November in New Zealand, however ‘Hort7709’ is not ready for training until mid November. The main shoot of the vine, when mature, has a medium number of thorns, an absent or very weak node pubescence and a reddish-purple stripe.
Laterals: Average 72 cm in length which is considered relatively long, they are predominantly distributed in the top third of the vine, which accounts for the club-shape, in a spreading attitude, and carry a medium number of cones.
Leaves: Overall shape palmatified, with an average of three lobes which ranged from slightly to strongly overlapping. The average length of the lobes was 116.7 mm. The top side of the leaves were coloured between near green 137A-B, the underside was between near green 137C and 138A, with veins near yellow green 146C-D. The colouration of the veins extended down the underside of the petioles which averaged 68 mm in length and 4.2 mm in width. The rest of the petiole was coloured near greyed purple N186C, this grew slightly lighter towards the leaf end. The margins of the leaves were acutely denate. The topside of the leaves had evenly distributed small white straight trichomes; these were also found along the main veins on the underside of leaves. The petioles had had raised lumps the same colour as the surrounding tissue topped with bifid transparent trichomes.
Cone physiology: Cones are small in size, with slightly open bracts of a medium length. The cones mature late in the season, 21st-30th March.
Chemistry profile: ‘Hort7709’ has undergone extensive chemistry profile testing and the following are six year data averages (FIG. 1). It is compared with ‘Pacifica’ and ‘NZ Hallertauer’ as these are commonly grown aroma hop cultivars in New Zealand. The chemistry profile of ‘Hort7709’ differs significantly from both ‘Pacifica’ and ‘NZ Hallertauer’ in a number of ways; these include lower content of alpha acids when compared to ‘NZ Hallertauer’ and ‘Pacifica’, significantly higher percentages of farnesene than either comparator. The proportion of essential oils at harvest found in ‘Hort7709’ cones differs considerably from that found in either ‘Pacifica’ or ‘NZ Hallertauer’ as does the overall oil content (mg/100 g). Consequently ‘Hort7709’ delivers a unique flavour, aroma, and bittering quality when processed and is therefore considered to be distinct.
Use: Flavouring and bittering ingredient for beer in the aroma market.

We claim:

1. A new and distinct hop plant substantially as illustrated and described.
